(19) United States
(12) Patent Application Publication
(10) Pub. No.: US 2017/0109471 A1
(43) Pub. Date: Apr. 20, 2017

(54) VARIANT ANALYSIS IN HIGH-THROUGHPUT SEQUENCING APPLICATIONS

(71) Applicant: VELA OPERATIONS SINGAPORE PTE. LTD., Singapore (SG)

(72) Inventors: Pramila ARIYARATNE, Singapore (SG); Charlie LEE, Singapore (SG)

(73) Assignee: VELA OPERATIONS SINGAPORE PTE. LTD., Singapore (SG)

(21) Appl. No.: 15/120,193

(22) PCT Filed: Feb. 19, 2015
(86) PCT No.: PCT/IB2015/051278

§ 371 Date: Aug. 19, 2016

(30) Foreign Application Priority Data
Feb. 20, 2014 (GB) 1402996.1

(51) Int. Cl. G06F 19/22; C12Q 1/68

(52) U.S. Cl. CPC G06F 19/22; C12Q 1/6874

(57) ABSTRACT

The present invention relates to methods of determining, identifying, detecting and annotating nucleic acid sequences suspected in a sample. Particularly, the methods of the invention permit annotating nucleic acid variants present in a sample. The methods are based on data obtained in high-throughput sequencing of nucleic acids obtained from a sample. The invention relates also to computer programs capable of executing the inventive methods and apparatus suitable of running respective computer software.

The present invention relates to methods and products in the field of diagnostic applications, preferably clinical diagnosis using high-throughput sequencing. The methods allow determining the presence or absence of wild-type nucleic sequences and variants thereof as well as quantifying the frequency of wild-type and/or variant nucleic acid sequences. The invention further allows improved methods of detecting the occurrence and/or frequency at which the variant nucleic acid sequences encode amino acid sequences that comprise modified amino acid residues when compared with wild-type amino acid sequences.

### BACKGROUND

In the field of clinical diagnosis it can be important to know exactly, whether or not a given target sequence, e.g. a disease-associated nucleic acid sequence (e.g. an oncogene or a nucleic acid derived from a pathogen) is present or absent. Further, it is sometimes, important to know the frequency of occurrence of certain target nucleic acid sequences.

Methods of detecting nucleic acid sequences of interest relying on PCR have been used in recent years to a great extent to detect target nucleic acids. There are situations, however, where PCR is not sufficiently accurate in the detection of target nucleic acids, for example when nucleic acids should be detected that exist in different variants such as genes that are involved in the development of cancer or genes derived from viruses that exist as variants of wild-type sequences. KRAS is an example for a gene that is implicated in the development of many different human cancers. The KRAS gene exists in various isoforms characterized by nucleic acid mutations at several distinct locations of the gene. These nucleic acids mutations, most of them are single nucleotide mutations (SNPs), can result in nucleotide codon changes encoding amino acid residues that differ from the wild-type amino acid residues. Such mutations can be associated with an altered phenotype of the protein in terms of its activation status or its potential of being inhibited by certain drugs.

To provide another example, it is known that certain viruses, such as HCV or HIV exist in a large number of genotypes. These genotypes differ from each other both, on the level of nucleic acid sequences and on the level of amino acid sequences. For certain therapeutic applications, it is important to know the viral genotype(s), because mutations in viral proteins can be associated with altered susceptibility or resistance to anti-viral drugs, e.g. interferons or drugs used in highly active anti-retroviral therapy (HAART) of HIV-infected individuals involving the administration of viral protease inhibitors or inhibitors of the viral reverse transcriptase.

When nucleic acids are isolated from samples, e.g. clinical samples, and analyzed by PCR, the sensitivity of the method is sometimes not sufficient to detect different nucleic acid sequences. Primer dimerization particularly in multiplex PCR methods, insufficient binding of primers or probes to different or so far unknown isoforms and large amounts of materials, e.g. primers and probes, to detect all known nucleic acid isoforms of a target sequence pose problems in diagnostic applications. Further, the costs and efforts for setting up and conducting a large number of PCR reactions to identify a multitude of different nucleic acid sequences is time-consuming, laborious and error-prone.

These disadvantages can be overcome when high-throughput sequencing, also referred to as “Next Generation Sequencing” (NGS), is used. NGS is getting increasingly reliable and less expensive due to progress in automatization of the sequencing process, preparation of samples to be sequenced, and data analysis. NGS provides enormous amounts of sequencing data in relatively short time. Further, NGS allows with high accuracy the detection of different isoforms of nucleic acid sequences. When nucleic acids are analysed using NGS, a plurality of previously amplified target sequences are sequenced that cover the target sequence of interest multifold. For example, when the target nucleic acid is a viral gene, nucleic acids isolated from a sample are first amplified, e.g. using PCR, and a plurality of the obtained PCR-sequences are sequenced.

When the oligonucleotides to conduct a PCR are selected in such manner that several isoforms of a target nucleic acid sequence are co-amplified, the amplification product contains also a plurality of different isoforms. Sequencing of the PCR products will not only allow determining the identity of the amplified nucleic acid sequences, but it will also allow determining whether or not a particular sequence of interest is present or absent. Finally, sequencing permits also determining the frequencies of occurrence of one or more different nucleic acid isoforms in a sample.

For example, when a sample contains 5% of isoform A, 30% of isoform B, and 65% of wild-type nucleic acid sequence (C), the NGS based analysis will provide not only information on the nucleic acid sequences that were obtained from a sample. NGS will also allow determining the percent frequency at which said nucleic acids are present in a sample. Such information may be important, e.g. in choosing the right therapy for a patient from whom the clinical sample was obtained.

A problem exists however in the correct analysis of the obtained nucleic acid sequence information. As mentioned earlier, NGS provides several thousand sequence reads of different lengths, sequences that may contain partially non-coding nucleic acid sequence fragments, and sequences that so far were not known, e.g. sequences comprising novel single or multiple nucleotide mutations, insertions, deletions, etc. Therefore, a need exists to correctly and efficiently analyze the obtained data, to align fragments, to exclude irrelevant information, and to recognize relevant and new nucleic acid sequence isoforms rather than disregarding the same as artifacts.

NGS consists essentially of four steps:
Collection of sample material, isolation and purification of nucleic acids;
Generation of templates, e.g. double-stranded DNA, reverse-transcribed RNA to obtain cDNA, provision of a sequencing library comprising fragmentation of the DNA, size selection and oligonucleotide adapter ligation. Depending on the NGS technology used, a library is either sequenced directly (single-molecule templates) or is clonally amplified before sequencing;
Sequencing of the previously produced sequencing library in parallel sequencing reactions, e.g. pyrosequencing, ion semiconductor sequencing, etc.;
Post-sequencing data analysis.

According to Rizzo and Buck (Key Principles and Clinical Applications of “Next Generation” DNA sequencing, Cancer Prev Res (Phila), 2012 July; 5(7):887-900) one key limitation of NGS is the high volume of data that has to be analyzed. As NGS reactions generate sequence data sets in the range of megabases to gigabases, the analysis requires highly elaborated information technology at all stages of sequencing, data tracking, storage, and quality control. These extensive data gathering capabilities are described by Rizzo and Buck (supra) as shifting the bottlenecks in genomics research from data acquisition to those of data analysis and interpretation. NGS machines generate data at such a rapid pace that there is a constant need for analytic approaches capable of analyzing such data sets. In general, the initial analysis (also referred to as “base calling”) is conducted by proprietary software on the sequencing platform. After base calling, the sequencing data are aligned to a reference genome if available or a de novo assembly is conducted (Rizzo and Buck, supra). Sequence alignment and assembly is an active area of computational research with new methods being developed (Flicek and Birney, Sense from sequence reads: methods for alignment and assembly. Nat Methods 2009; 6: S6-12). Once the sequence is aligned to a reference genome, the data need to be analyzed. The quality and quantity of sequence data derived from NGS experiments will ultimately determine how comprehensive and accurate downstream analyses can be.

Qualitatively, individual base calling error rates vary between NGS platforms. All NOS platforms provide confidence scores for each individual base call, enabling researchers to use different quality filters when mining their sequence data. Quantitatively, the amount of sequence data can be assessed by the metric of sequencing “coverage.” (Rizzo and Buck, supra) As used herein, sequence coverage (also called “depth”) refers to the average number of times a base pair is sequenced in a given experiment. Qualitatively, uneven sequence coverage can also interfere with the analysis of sequence variants. A deeply sequenced sample with nonuniform read distribution can still leave a substantial portion of the genome unsequenced or undersequenced, and analysis of these regions will not be able to identify sequence variations such as single-nucleotide polymorphisms (SNP), point mutations, or structural variants, because these locations will either be unsequenced or confounded by sequencing errors (Rizzo and Buck, supra). Ultimately, coverage depth, distribution, and sequence quality determine what information can be retrieved from each sequencing experiment.

For discovery of structural variants (e.g., insertions, deletions, translocations), accurate identification of a complete human genome sequence with current (second-generation) platforms, requires approximately 20× to 30× sequence coverage to overcome the uneven read distributions and sequencing errors (Thompson J F, Milos P M, The properties and applications of single-molecule DNA sequencing Genome Biol. 2011; 12(2):217. doi: 10.1186/gb-2011-12-2-217).

The above shows that the analysis of data obtained by NGS sequencing methods is a critical issue. The present invention relates to methods of analysing such data, in particularly in the detection of the presence or absence of a nucleic acid of interest, e.g. a particular isoform of a nucleic acid sequence. Therefore, the methods of the invention may be used in the diagnosis of certain diseases or disorders associated with the presence of particular isoform of a gene, e.g. an oncogene, or with a gene of a pathogen, which may be associated with the presence and severity of certain diseases, e.g. genes of S. aureus that are associated with resistance to certain antibiotics. Further, the methods of the invention allow for detection of previously unknown nucleic acid sequence isoforms. The methods of the invention permit also identifying with high reliability and accuracy whether or not a sample contains nucleic acid sequence isoforms that encode amino acid sequences with alterations when compared with wild-type sequences. The methods of the invention relate also to therapeutic applications of the thus evaluated data, e.g. whether or not a given patient may be treated with a specific drug depending on the interpretation of the result of the analysis. For example, if the analysis of the sequencing data results in the finding that an amino acid residue is mutated, the physician may base the following therapy on this finding, i.e. the presence or absence of a given amino acid mutation can suggest that a therapy with a certain drug, will be successful or not (e.g. refer to Genentech patents). Further, if as the result of an analysis of sequencing data the presence or absence of a viral genotype (e.g. of HCV or HIV) is confirmed, the physician in charge can decide whether or not a certain virostatic drug (e.g. gamma interferon, RT-inhibitor, protease-inhibitor, etc.) may be used with a reasonable expectation of success. The invention provides solutions to the above tasks. Other aspects of the invention will be explained in the detailed description.

### DETAILED DESCRIPTION

As used in the specification and the claims the singular forms of “a” and “an” also include the corresponding plurals unless the context clearly dictates otherwise.

The term “about” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only.

The term “detecting the presence” as used herein is to be understood in the meaning of “detecting the presence or absence”. As mentioned in the method as claimed in the present application, the sample to be analyzed is suspected to comprise a nucleic acid comprising, a consensus nucleic acid sequence (which may also be designated as target sequence) indicative of the presence of a pathogen.

In the context of the present invention the expression “target sequence” designates, e.g. a genomic region that is specific for a pathogen. Alternatively, the target sequence may be an oncogene, or it may be a gene that is known for its role in disease. For example, there are mutations that are involved in the predisposition to certain inflammatory, autoimmune diseases, or metabolic diseases or disorders.

As an example of a pathogen, the presence and target sequences of which should be determined, Hepatitis C Virus can be mentioned. In the methods of the present invention, it is taken into account that more than one nucleic acid sequence variants of HCV exist, i.e. more than one genotype, subtype or strain of said pathogen. For example, the NS5B genomic region of HCV allows identifying the presence of HCV in a sample. However, several genotypes and subtypes of this genomic region exist, i.e. while the genomic region comprises a consensus sequence indicative of all HCV genotypes these individual genotypes and subtypes have different nucleic acid sequences or variants of said nucleic sequences. Important target nucleic acids analyzed in the methods according to the present invention are viral or microbial, e.g. bacterial genes that influence the course of infection, the microbial virulence and/or pathogenicity, and/or the resistance towards anti-viral drugs, antibiotics, etc.

In the context of the present invention the term “nucleic acid” refers to a naturally occurring deoxyribonucleotide or ribonucleotide polymer in either single-or double-stranded form. The nucleic acid may particularly be double-stranded DNA and single-stranded RNA.

The term “sequence” as used herein refers to the sequential occurrence of the bases in a deoxyribonucleotide or ribonucleotide polymer, wherein a base found in a deoxyribonucleotide polymer is selected from the group consisting of A, T, G and C and a base found in a ribonucleotide polymer is selected from the group consisting of A, U, G and C. A sequence of bases in a deoxyribonucleotide polymer may thus e.g. be GGAAGCAAGCCT, whereas a sequence of bases in a ribonucleotide polymer may e.g. be GGAAUCGAU.

As used herein, the term “sample” refers to any biological sample from any human or veterinary subject that may be tested for the presence of a nucleic acid comprising a target sequence. The samples may include tissues obtained from any organ, such as for example, lung tissue; and fluids obtained from any organ such as for example, blood, plasma, serum, lymphatic fluid, synovial fluid, cerebrospinal fluid, amniotic fluid, amniotic cord blood, tears, saliva, and nasopharyngeal washes. As listed above, samples may also be derived from a specific region in the body, e.g. the respiratory tract; samples from the respiratory tract include throat swabs throat washings, nasal swabs and specimens from the lower respiratory tract.

The sample may in particular be derived from a human or a veterinary subject. Accordingly, a “patient” may be a human or veterinary subject. If reference is made to a “clinical sample”, this indicates that the sample is from a patient suspected to be infected by a pathogen having a nucleic acid comprising a target sequence.

As used herein, the term “amplification” refers to enzyme-mediated procedures that are capable of producing billions of copies of nucleic acid target. Examples of enzyme-mediated target is amplification procedures known in the art include PCR. Amplification of a genomic region, e.g. using (RT-) PCR, and sequencing of the amplification product as well as the subsequent analysis using the methods of the invention allows determining whether or not a specific genetic mutation, which may be known or unknown, or a wild-type of a target nucleic acid is present in a sample from which the amplified nucleic acid was obtained.

A “PCR reaction” has first been described for the amplification of DNA by Mullis et al. in U.S. Pat. No. 4,683,195 and Mullis in U.S. Pat. No. 4,683,202 and is well known to those of ordinary skill in the art. In the PCR technique, a sample of DNA is mixed in a solution with a molar excess of at least two oligonucleotide primers of that are prepared to be complementary to the 3′ end of each strand of the DNA duplex (see above, a forward and a reverse primer); a molar excess of nucleotide bases (i.e., dNTPs); and a heat stable DNA polymerase, (preferably Taq polymerase), which catalyzes the formation of DNA from the oligonucleotide primers and dNTPs. Of the primers, at least one is a forward primer that will bind in the 5′ to 3′ direction to the 3′ end of one strand (in the above definition the non-sense strand) of the denatured DNA analyte and another is a reverse primer that will bind in the 3′ to 5′ direction to the 5′ end of the other strand (in the above definition the sense strand) of the denatured DNA analyte. The solution is heated to about 94-96° C. to denature the double-stranded DNA to single-stranded DNA. When the solution cools down and reaches the so-called annealing, temperature, the primers bind to separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. As the sequence being amplified doubles after each cycle, a theoretical amplification of a huge number of copies may be attained after repeating the process for a few hours; accordingly, extremely small quantities of DNA may be amplified using PCR in a relatively short period of time.

Where the starting material for the PCR reaction is RNA, complementary DNA (“cDNA”) is synthesized from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR”.

In the context of high-throughput sequencing as used herein, amplification of nucleic acids that were extracted from a sample can be performed using the Ion AmpliSeq method (Life technologies, Inc.), which are known to technical experts in the field of high-throughput sequencing (or Next Generation Sequencing) may be cited. Of course, it is possible also to use other methods for the amplification of nucleic acids derived from a sample. The obtained sequence information is subsequently analyzed using the methods and devices disclosed herein.

The term “sequencing” is used herein in its common meaning in molecular biology. Thus, the exact sequential occurrence of bases in a nucleic acid sequence is determined.

The term “oncogene” is used herein in its common meaning in molecular biology and oncology, respectively. Thus, there are e.g. mutations known in genes, which render a “normal or wild-type” gene oncogenic, i.e. cancer-inducing; examples in this respect are mutations rendering kinases constitutionally active such that specific signals (e.g. growth inducing signals) are constantly signaled and corresponding processes initiated. “Oncogenes” as used herein may also relate to intra- or inter-chromosomal translocations resulting also in cancer-inducing situations. In the methods used in the context of the present invention, oncogenes causing or involved in the development of cancer or neoplasia in humans are preferably targeted. More particularly, the methods are suitable for the detection, determination and annotation of nucleic acid variants that are involved in development of resistance of a cancer towards a specific drug. The methods of the present invention thus allow determining and annotating sequence variants of oncogenes, This information can form the basis of a therapeutic decision through the treating physician, e.g. whether or not the administration is of any clinical benefit for the patient whose sample is analyzed, i.e. from which the nucleic acids that are targeted and investigated are derived.

The term “pathogen” as used herein is used in its broadest meaning. Thus, a pathogen may be any type of bacteria, archaeum, protozoum, fungus and virus. It is explicitly mentioned that viruses fall under the definition of a “microorganism” as used herein.

A genomic variant is defined as any difference between the sequenced sample and a reference genome. However, the methods of the invention, are suitable to identify, detect small scale variants such as Single Nucleotide Polymorphisms (SNPs), Multiple Nucleotide Polymorphisms (MNPs) and small (˜<500 bp) Insertions and Deletions.

Depending on specific genomic variants present in individuals, various drugs (for cancer, for example) may respond differently. Therefore before any treatment is, carried out, it is preferably to test for specific mutations (variants) that may affect drug response.

COSMIC (Catalogue Of Somatic Mutations In Cancer) represents the largest such database containing to mutations specific to cancer. However since the drug response is typically associated with a protein change, databases such as COSMIC are annotated with protein change and coding sequence, rather than genomic change. A big problem is therefore annotating genomic variants with COSMIC data.

This is, inter alia, due to the fact that some COSMIC annotated protein changes may span across multiple neighboring basepairs or codons in coding sequence space. As single codons may span across two exons, the two neighboring basepairs in coding sequence space maybe actually be a few hundred base pairs apart in genomic space. Furthermore some mutations could be subsets or supersets of another mutations.

For instance in the mutation V600E (Amino acid ‘V’ in position 600 in protein sequence change to amino acid ‘E’) in gene BRAF which is associated with skin cancer, the reference base ‘T’ in coding sequence position 1799 is mutated in to base ‘A’. (This is denoted as c.1799T>A in codon space). However there exists another known mutation V600K (c.1798_1799GT>AA) which occurs when G becomes A at position 1798 AND T becomes A at position 1799. The first case is in fact a subset of the second case. However when annotating mutations, the two cases need to strictly separated as a patient with mutation V600E might need to be treated with a drug different from that is used to treat with someone with V600K.

It is also noted that it is possible that some annotated mutations exist where the two mutated positions are not strictly adjacent. For instance there could exist a case c.1797_1799AGT>CGA. Position 1798 has not mutated and will not be picked up by a variant caller. Another possible complication is that two base positions in coding sequence 1798 and 1799 are actually in two different exons, and therefore can be far apart by a few hundred bases in the genomic sequence.

All SNP annotating methods or computer programs, e.g. also those of the present invention are preceded by two steps. First a mapping program such as BWA or TMAP maps sequenced reads to a reference genome. Secondly a variant caller program such as GATK or Torrent Variant Caller uses this mapping information to call variants in the sample. Here a variant could be any small scale difference between the reference genome and the sample genome, such as Single Nucleotide Polymorphisms (SNPs), Multiple Nucleotide Polymorphisms (MNPs) or small Insertions and Deletions.

The results of the variant caller are usually presented as a VCF (Variant Caller Format) file. It lists all positions in the reference genome where a variant is observed and a summary of information about each variant (Reference sequence, Variant sequence, No. of sequencing reads, etc.). This VCF file is the starting point for all variant annotation programs.

However by starting from VCF file (and ignoring preceding information), a variant annotation program may lose crucial information. Variant caller programs differ in how they treat adjacent variants. Most of them will call each position individually and will not consider the fact that some variants may occur together. There are programs that will group all adjacent variants to a single superset, but will not consider that fact there could be some cases (portion of the reads/fraction of the sample) in which only 1 of the grouped mutations occur. Furthermore, if the two mutations are apart (either one or two bases or a few hundred bases in case of different exons), variant calling programs will not group such calls together.

The only reliable way to identify such co-occurrences within multiple SNP calls is the refer back to the original read sequences. The methods and computer tool of the present invention use both VCF file and the original mapping output to regroup all SNPs and MNPs and reports exactly which combinations of mutations occur together along with perceived mutation rate (percentage of occurrence within sample). As a two step approach is taken, first in genomic space and then in coding sequence space, the methods of the invention are also capable of grouping variants which are far apart in genomic space, but adjacent or neighboring in coding sequence and protein space.

Other existing SNP annotation programs such as SnpEff or SNP-nexus (http://www.snp-nexus.org/) do not refer back to the original mapping data and therefore limited by the information provided by the VCF file.

Therefore, in one aspect the invention relates to a method comprising the following steps:
a) Providing genomic locations of regions of interest (e.g. amplicon regions in amplicon sequencing);
b) Providing a mapped BAM file using software suitable for sequence alignments (e.g. TMAP, BWA) and a list of genomic variants in VCF format using a software suitable for variant calling (e.g. Torrent Variant Caller, GATK) for a sample;
c) Filtering, of variant calls in provided VCF that are not within the provided genomic locations of regions of interest;
d) Determining co-occurring and mutually exclusive mutations from list of variant calls in provided VCF and mapped BAM file;
e) Determining the coding DNA sequence and amino acid change of mutations including mutations across introns;
f) Annotating mutations with COSMIC database.

One purpose of the methods of the invention is therefore to match a given variant/set of variants with possible cosmic annotations, as most actionable targets are referred to by their cosmic annotations. Actionable targets are variants of a targeted nucleic acid sequence that encode altered amino acid sequences, wherein said alteration results in a phenotype of interest, e.g. a mutation in a polypeptide encoded by an oncogene that is more resistant or susceptible to a given drug)

Therefore, the methods of the invention take an approach to maximize the chance that a cosmic annotation can be found for a given variant. To this end, initially each variant is compared against cosmic entries at genomic level. The cosmic VCF file lists all cosmic entries with their corresponding genomic variant. Should any of the sample variants match a cosmic VCF entry the sample variant is annotated with that cosmic entry. If no cosmic match was found at genomic level, each mutation is translated to coding and amino acid the inventive methods comprise a step wherein a search is conducted for a cosmic match at gene/coding sequence change level. The entire process is explained below.

The methods of the invention fulfill amongst other at least one of many purposes. Given a list of genomic variations exist, the methods:
i. Filter out variations irrelevant to a given analysis;
ii. Determine which variants in a given neighborhood co-occur;
iii. Cross-check each variant against a database comprising known target sequences (e.g. COSMIC);
iv. Annotate sequences with respective information found in said database comprising known target sequences and/or identifying new variants;
v. When the variant affects coding sequence of a targeted gene, the coding sequence change and amino acid change is predicted;
vi. Compare each variant against a list of predetermined target variants and annotate if it is present in the list.

The present invention relates to a method of determining the presence of a target nucleic acid sequence, or a nucleic acid sequence of interest, in a sample. In a preferred aspect, the method is an analytical program comprising a respective algorithm. Further, the invention relates to devices (e.g. apparatus) comprising software that is suitable to execute methods of the invention.

In another aspect, the above methods of determining the presence of a target nucleic acid sequence or a nucleic acid sequence of interest in a sample are characterized by post-sequencing analysis of the nucleic acid sequence, wherein the analysis comprises the detection and/or quantitation of variants in the nucleic sequences obtained from the sample.

In particular, the methods of the invention allow determining the presence of a nucleic acid sequence in a sample, wherein said nucleic acid sequence comprises at least one variant nucleotide when compared with nucleic acid sequences comprising as a reference at least one wild-type nucleic acid sequence.

Further, the methods of the invention allow determining the presence of a nucleic acid sequence obtained from the sample, wherein the sequence may be compared with at least a wild-type nucleic acid sequence and with variants of said wild-type sequence comprising at least one variant nucleotide residue when compared with the said wild-type (e.g. oncogenes, viral genes that are associated with certain phenotypes such as susceptibility or resistance to a drug, etc.).

In some aspects of the above described methods the variant nucleic acids encode mutant polypeptides that are associated with a phenotype of interest, e.g. increased or decreased susceptibility or resistance towards certain drugs (e.g. anti-cancer drugs, antibacterial drugs or virostatic drugs, etc.). Such drugs may be selected from known drugs, e.g. small molecules, antibiotics, protease inhibitors, reverse transcriptase inhibitors, cell-signaling molecules, such as interferons, antibodies, etc.

The methods of the invention either follow or comprise steps required for high-throughput sequencing (also referred to as Next Generation Sequencing). In preferred embodiments, the invention comprises ion semiconductor sequencing, e.g. as provided by the technology proprietary to Ion Torrent.

At the onset of the NGS procedure, it is required to obtain a sample suspected to contain nucleic acids of interest, i.e. target nucleic acids. Nucleic acids can be extracted from the sample using methods known in the art. Preferably, the extraction of nucleic acids is performed in a (semi-) automated system.

Further in the NGS procedure either preceding the methods of the invention or comprising the same, the genomic locations of regions of interest (e.g. amplicon regions) are provided.

In further aspects of the methods of the invention, the genomic locations of the regions are known as are the locations of the primer binding sites (i.e. their coordinates). The primers define the target regions of interest. This information serves as input to analysis methods comprising a computer program as it speeds up the program as it can now specifically focus on analyzing variants called within the regions of interest. Further, in the methods of the invention, for each target region (amplicon), the algorithm determines which bases are in exon regions, intron, regions and intergenic regions for the target gene transcripts. Methods of the invention, computer program and apparatus comprising such programs or executing the methods of the invention can comprise NGS steps or follow the actual NGS steps.

Accordingly, the inventive methods and devices comprise the step of using a software that provides variant calls in the form of VCF format (e.g. Torrent Variant Caller, GATK). Subsequently, the inventive method (or computer program) analyzes these variant calls based on the initial sequencing data and external database sources to provide a more accurate result.

The methods of the invention in a further step comprise providing a mapped BAM file. This file has a binary format for storing sequence data. A BAM file (.bam) is the binary version of a SAM file. A SAM file (.sam) is a tab-delimited text file that contains sequence alignment data, in a human readable format. Both specifications are described in http://samtools.sourceforge.net/SAMv1.pdf.

The inventive methods may further comprise a step wherein variant calls provided in VCF format that are not located in the genomic regions of interest (or in the target sequence) are filtered off.

In the inventive methods or computer programs comprise steps of determining co-occurring and mutually exclusive mutations in the list of variant calls provided in VCF and/or the mapped BAM file. In the inventive methods, co-occurring mutations are Multiple-Nucleotide-Polymorphisms (MNP) that occur in the same cell. Mutually exclusive mutations are neighboring Single-Nucleotide-Polymorphisms (SNP) that do not occur in the same cell.

For e.g., when the sequence below is taken as reference sequence, the underlined nucleotides tg are those where variants can occur.

1)
cagtcgatcgatcgactgcgattgtgtgctagcatgcatcgatcgaga

For illustrative purposes it is supposed that the nine reads below were obtained in a NGS run, which variants highlighted in bold-faced capital letters:

1)
cagtcgatcgatcgactgcgatACtgtgctagcatgcatcgatcgaga
2)
cagtcgatcgatcgactgcgatACtgtgctagcatgcatcgatcgaga
3)
cagtcgatcgatcgactgcgatACtgtgctagcatgcatcgatcgaga
4)
cagtcgatcgatcgactgcgatAgtgtgctagcatgcatcgatcgaga
5)
cagtcgatcgatcgactgcgatAgtgtgctagcatgcatcgatcgaga
6)
cagtcgatcgatcgactgcgatAgtgtgctagcatgcatcgatcgaga
7)
cagtcgatcgatcgactgcgattCtgtgctagcatgcatcgatcgaga
8)
cagtcgatcgatcgactgcgattCtgtgctagcatgcatcgatcgaga
9)
cagtcgatcgatcgactgegattCtgtactagcatgcatcgatcgaga

The first 3 reads, 1) to 3), indicate that “AC” is a MNP that occur together. The next 3 reads, 4) to 6) indicate that there is also an “A” SNP that occurs by itself. The last 3 reads, 7) to 9) indicate that there is also a “C” SNP that occurs by itself. Hence, the “A” SNP occurs mutually exclusive to the “C” SNP. In programs known in the art, e.g. in the Ion Torrent Variant Caller, the details here are lost and the summarized variant call result “AC” with a support of 9 reads is given. In the inventive methods, the details for all 9 reads are given, i.e. “AC”—3 reads, “A”—3 reads, and “C”—3 reads.

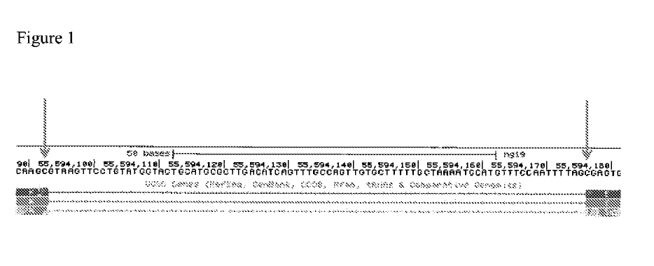

The methods of the invention may further comprise determining the coding DNA sequence and amino acid changes due to variants in the nucleic acid sequence. The methods of the invention may also comprise a step allowing determining, mutations in the amino acid sequence that are encoded by nucleotide variants present on different exons. That is, when variants are found in different exons, after splicing out the intron between these exons, an encoded amino acid may be mutated, e.g. when the last nucleotide of one intron and the first nucleotide of another intron are mutated. When exons are spliced out, the two variant nucleotides are adjacent in a nucleotide sequence and may encode a different amino acid residue in the encoded polypeptide. Such alterations in the amino acid sequence and in the encoded nucleotide sequence may be determined in the methods of the present invention, which is not the case in any methods for the determination of variants known to the applicant. In more detail, due to existence of introns and exons in some genomic target sequences, it is possible that two bases are not adjacent in genomic location, but adjacent in coding sequence location. FIG. 1 illustrates this. Therein two bases marked with arrows are far apart in genome, but since they are separated by an intron, in the coding space they are adjacent and could fall within same codon or adjacent codons. Therefore when grouping mutations together it should be done relative to coding sequence position (when within coding region) as it could affect the same or adjacent codons.

Therefore, the herein provided methods are an advantage when compared with methods that are incapable of annotating variants resulting in mutations in the amino acid sequence. Contrary to the methods of the present invention, prior methods comprise two distinct steps. The first step is directed to variant calling, and the second step is the annotating step. In those methods, the annotating program does not have access (or does not make use of) underlying sequencing data. Therefore, such methods cannot determine whether or not two variants co-occur (regardless whether they are next to each other in two different exons). Prior methods will treat mutations exactly as they are reported by the variant caller. Variant caller methods will usually report adjacent variants together. However if variants are separated by a large distance (e.g. when they are in two different exons), prior art variant caller methods will report them separately (as it does not make use of Gene/Exon data).

The end result of prior methods is that they annotate two mutations in two different exons separately and thus ignore the combined effect of them.

The methods of the present invention prevent the above mentioned inaccuracies by looking at the underlying sequencing data to determine if two mutations across two exons are related, and if so annotate them together.

The methods of the invention annotate variants with a database, e.g. COSMIC. The methods of the invention may also comprise the provision of a report of the results obtained in the variant analysis. Any variant not matching a COSMIC ID would be considered as a novel variant and is reported as it is (without COSMIC annotation). Therefore, the methods of the invention are not only relating to methods of determining sequences of target sequences in nucleic acids obtained from a sample, but also relating to the identification of novel variants of the target nucleic acid. In both cases, the methods may also provide the amino acid sequences of the nucleic acids obtained from the sample. Methods of identification or determining the sequences of novel nucleic acid variants of a target sequence will be helpful in assisting the treating physician to make therapeutic decisions. Further, the identification and annotation of novel variants of target sequences may help developing specific drugs, e.g. antibodies targeting the encoded variant polypeptides. For example, when a novel variant nucleic acid sequence encodes a mutant amino acid sequence, specific antibodies may be generated against the mutant polypeptide, e.g. neutralizing antibodies, blocking antibodies, etc. Further, on the basis of the knowledge of the encoded new polypeptide, structural changes in the polypeptide may be determined using methods known in the art. Knowledge of the polypeptide structure may assist in designing small molecules affecting the function of the encoded polypeptide, e.g. as in case of the drug Imatinib, which selectively blocks the ATP-binding site in various tyrosine kinases.

In the inventive methods of determining and/or annotating and/or identifying variants, the data input consists of
a) A list of genomic variants in VCF format;
b) A Mapped BAM file (sorted and indexed) that was used to make the variant calls;
c) An ‘Assay file’ containing sequencing data obtained in the experiment, e.g. the NGS run, wherein the assay file contains information on the genomic location of each amplicon and its sequence, a list of targeted genes and information on their exons or exon-intron-structure, and transcript sequences (from ensembl/used by COSMIC), a list of mutations targeted in an experiment (with cosmic IDs, coding sequence change, amino acid change if applicable);

d) COSMIC VCF files, wherein said COSMIC VCF files contain variant entries (in VCF format) for each VCF entry, annotated with coding sequence and amino acid change (it is noted that COSMIC files can be downloaded from the COSMIC ftp site (ftp://ngs.sanger.ac.uk/production/cosmic/).

Ensembl referred to above in step c) is a joint scientific project between the European Bioinformatics Institute and the Wellcome Trust Sanger Institute, which was launched in 1999 in response to the imminent completion of the Human Genome Project. Ensembl is one of several well known genome browsers for the retrieval of genomic information (http://www.ensembl.org/index.html).

In the above item b), the sort command sorts a BAM file based on its position in the reference, as determined by its alignment. The index command creates a new index file that allows fast look-up of data in a (sorted) SAM or BAM file. A BAM file contains the alignment information of reads generated from a sequencing run. The alignment is obtained from mapping the reads to a reference genome.

The invention is explained in more detail in the example below.

### EXAMPLE 1

1) Filtering Variants
In a first step of the methods of the invention is to filter out variants that do not fall within amplicon regions as these calls are unreliable, e.g. all variants which are not completely contained within an amplicon region are not considered for any subsequent analysis. Thus, if part of a variant falls outside amplicon region, the entire variant will be filtered out.

2) Grouping Variants

The reasoning behind carrying out grouping step is as follows. Most known variant calling programs call variants at each base separately. For example if a variant caller may produce the following output,
Pos 1 A to T (50%)
Pos 2 C to G (50%)
When data are presented in such way, it is not clear whether the two mutations co-occur in a single cell. It could be that two mutations are mutually exclusive or 50% of the sample is wildtype and the remainder contains mutation Pos 1-2 AC to TG. Since this information is critical to determining cosmic annotation, the methods of the present invention group co-occurring variants together prior to matching with the COSMIC database.
Only SNPs and MNPs (Multiple Nucleotide Polymorphisms) are considered for grouping. All such variants that are adjacent to each other (in genomic location or in coding sequence location) form a potential group. For each potential group, all sequence reads which span all the variant positions are extracted. For example, it is assumed that the following 3 variants are detected:
X=Pos 1 A to T (11%)
Y=Pos 1 A to C (21%)
Z=Pos 2 T to G (41%)
The table below (Table 1) is filled based on how many reads exhibit each combination of the variants. Note that when counting reads, only reads with minimum phred score of 17 at variant position are considered.

TABLE 1
Example of variant distribution (Total reads: 1000)

| | | | |
|---|---|---|---|
| X | Y | Z | Read count |

| | | | | |
|---|---|---|---|---|
| Yes | Yes | Yes | 0 | 0% |
| Yes | Yes | No | 0 | 0% |
| Yes | No | Yes | 5 | 1% |
| Yes | No | No | 95 | 10% |
| No | Yes | Yes | 200 | 20% |
| No | Yes | No | 5 | 1% |
| No | No | Yes | 195 | 20% |
| No | No | No | 500 | 50% |

Table 1 shows that 50% of the sample is wild-type (No/No/No) while other combinations occur at varying frequency. When a minimum 5% detection threshold is targeted, any combination that occur at less than 5% frequency is filtered out. Based on the above table, the methods of the invention determine that variants occur in following groupings: X, YZ, Z. Instead of reporting variants as listed above (X=Pos 1 A to T . . . etc), variants are reported as follows in a form that accurately portray their co-occurrence for use in downstream analysis. It is noted that variant combinations that occur at 1% frequency are ignored
Pos 1 A to T (10%)
Pos 1-2 AT to CG (20%)
Pos 2 T to G (50%)
Phred quality scores were originally developed by the program Phred to help in the automation of DNA sequencing in the Human Genome Project. Phred quality scores are assigned to each nucleotide base call in automated sequencer traces. Phred quality scores have become widely accepted to characterize the quality of DNA sequences, and can be used to compare the efficacy of different sequencing methods. Phred quality scores Q are defined as a property which is logarithmically related to the base-calling error probabilities P.

$$Q = - 10 {log}_{10} P$$

$$or$$

$$P = {10}^{\frac{- Q}{10}}$$

For example, if Phred assigns a quality score of 30 to a base, the chances that this base is called incorrectly are 1 in 1000. The most commonly used method is to count the bases with a quality score of 20 and above. The high accuracy of Phred quality scores make them an ideal tool to assess the quality of sequences. This is shown in Table 2 (Phred quality scores are logarithmically linked to error probabilities).

| Phred Quality | Probability of | Base call |
|---|---|---|
| Score | incorrect base call | accuracy |
| 10 | 1 in 10 | 90% |
| 20 | 1 in 100 | 99% |
| 30 | 1 in 1000 | 99.9% |
| 40 | 1 in 10000 | 99.99% |
| 50 | 1 in 100000 | 99.999% |

3) Determining CDS/AA Change
For SNPs and MNPs that are within coding regions, the coding sequence change and amino acid sequence change is inferred by the methods of the invention. Coding Sequence (CDS) change is determined by simple comparison of the coding sequence before and after mutation has taken effect. Amino acid (AA) changes are determined by comparing codon changes in the region with CDS changes. In order to adhere to COSMIC notation as much as possible, only codons with an amino acid change (non-silent) are listed unless they are flanked by codons with an AA change.

4) Annotating Cosmic Information
Once the co-occurring variants are grouped and resulting variant calls arc obtained, the basepair changes of each variant is compared against the entries in COSMIC VCF file(s). If a matching entry can be found in a COSMIC VCF file (matching entry is defined as one with same genomic location, reference bases and alternate bases) the variant is annotated with the matching COSMIC ID. In order to preserve the consistency with COSMIC database, the CDS change and amino acid (AA) change is copied from the COSMIC entry, rather than being inferred from genomic change,
Furthermore it is possible that different genomic mutations can result in the same net effect (hence same amino acid change).
For instance in the following sequence.

TTAGTGGAAGCC

deleting either AGTGGA (TTAGTGGAAGCC) or TGGAAG (TTAGTGGAAGCC) will result in the same sequence (TTAGCC). However the two deletions maybe considered two separate mutations in COSMIC and annotated with different COSMIC IDs. But during variant calling, the mutation is reported in only one of the two candidates above. In such instance, it is preferred to list all other COSMIC mutations that may result in the same ‘net’ genomic change. To facilitate this, an additional step was incorporated.
For each reported mutation, the ‘net effect’ in the flanking genomic region is constructed (e.g.: Del AGTGGA in TTAGTGGAAGCC results in TTAGCC). Similarly the ‘net effect’ of each COSMIC mutation in the database is constructed. Any Cosmic mutation that has the same net effect as the mutation in question is reported as a possible COSMIC annotation for said mutation. A single reported mutation could have multiple COSMIC annotations.
Reported mutations are again annotated against the Cosmic database based on Gene name and CDS changes. Any Cosmic mutation whose Gene name and CDS change matches with a reported mutation is annotated to that mutation.
   5) Annotating with Target List

Each processed variant is compared against a predetermined target variant list. A variant is considered a ‘target’ if either its COSMIC ID is present in the target list or if a target entry with same gene name and CDS change can be found in the list.

Any variant not matching a COSMIC ID would be considered as a novel variant and is reported as it is (without COSMIC annotation).

1. A method of annotating a variant in a nucleic acid sequence suspected to be present in a sample, comprising the steps:
a) selecting at least one nucleic acid sequence of interest,
b) using the isolated nucleic acids to provide templates for sequencing,
c) high-throughput sequencing of said templates;
d) providing a database comprising potential reference sequences,
e) grouping nucleotide variants in nucleic acid sequences to determine co-occurring and mutually exclusive mutations,
f) annotating variants identified in step e) using a sequence database
wherein the database referred to in d) comprises a wild-type sequence corresponding to the nucleic acid of interest,
wherein said method further comprises determining the genomic and coding DNA sequence in a single step, and
wherein the method further comprises filtering out variants that fall outside amplicon regions.

2. The method according to claim 1 wherein the database referred to in d) further comprises variants of the wild-type sequence.

3. (canceled)

4. (canceled)

5. (canceled)

6. (canceled)

7. The method according to claim 1, further comprising determining the encoded amino acid sequence.

8. A method of identifying a variant in a nucleic acid sequence suspected to be present in a sample, comprising the steps:
a) selecting at least one nucleic acid sequence of interest,
b) using the isolated nucleic acids to provide templates for sequencing,
c) high-throughput sequencing of said templates;
d) providing a database comprising potential reference sequences,
e) grouping nucleotide variants in nucleic acid sequences to determine co-occurring and mutually exclusive mutations,
f) annotating variants identified in step e) using a sequence database
wherein the database referred to in d) comprises a wild-type sequence corresponding to the nucleic acid of interest,
wherein the genomic and coding DNA sequences are determined in a single step, and
wherein said method further comprises filtering out variants that fall outside amplicon regions.

9. The method according to claim 8, wherein the database referred to in d) further comprises variants of the wild-type sequence.

10. (canceled)

11. (canceled)

12. (canceled)

13. (canceled)

14. The method according to claim 8, further comprising determining the encoded amino acid sequence.

15. The method according to claim 8, wherein the identified variant is selected from the group consisting of single nucleotide mutation (SNP), multiple nucleotide mutations, mutations encoded by nucleotide codons spanning introns, insertions, and deletions.

16. The method according to claim 1, further comprising the step of diagnosing a disease.

17. The method according to claim 1, wherein the disease is an infectious disease or a neoplastic disease.

18. The method according to claim 1, further comprising selecting a therapeutic treatment for the subject whose sample was analyzed.

19. (canceled)

20. (canceled)

21. A method of determining the presence or absence (frequency) of a nucleic acid isoform in a sample, comprising the steps:
a) selecting at least one nucleic acid sequence of interest,
b) using the isolated nucleic acids to provide templates for sequencing,
c) high-throughput sequencing of said templates;
d) providing a database comprising potential reference sequences,
e) grouping nucleotide variants in nucleic acid sequences to determine co-occurring and mutually exclusive mutations,
f) annotating variants identified in step e) using a sequence database.

22. A method of quantifying a nucleic acid sequence isoform in a sample comprising the steps:
a) selecting at least one nucleic acid sequence of interest,
b) using the isolated nucleic acids to provide templates for sequencing,
c) high-throughput sequencing of said templates;
d) providing a database comprising potential reference sequences,
e) grouping nucleotide variants in nucleic acid sequences to determine co-occurring mutually exclusive mutations,
f) annotating variants identified in step e) using a sequence database.

23. A method of detecting variants of a nucleic acid sequence in a sample, comprising the steps:
a) selecting at least one nucleic acid sequence of interest,
b) using the isolated nucleic acids to provide templates for sequencing,
c) high-throughput sequencing of said templates;
d) providing a database comprising potential reference sequences,
e) grouping nucleotide variants in nucleic acid sequences to determine co-occurring and mutually exclusive mutations,
f) annotating variants identified in step e) using a sequence database.

24. The method according to claim 21, wherein the variant has at least one single nucleotide variations compared with a reference wild-type nucleic acid sequence.

25. (canceled)

26. (canceled)

27. The method according to claim 21, wherein the variant encodes an amino acid sequence comprising at least one modified amino acid residue compared with a wild type amino acid sequence.

28. The method according to claim 21, wherein the variant encodes an amino acid sequence comprising at least one modified amino acid residue compared with a wild type amino acid sequence, and wherein the amino acid modification results in an altered susceptibility or responsiveness to a drug.

29. (canceled)

30. A software product comprising the software paths to carry out the steps of the method of claim 1.

31. (canceled)

32. (canceled)


SEQUENCE LISTING (5 sequences)

SEQ ID NO: 1 - DNA, 48 bp, Hepatitis C Virus
cagtcgatcgatcgactgcgattgtgtgctagcatgcatcgatcgaga

SEQ ID NO: 2 - DNA, 48 bp, Hepatitis C virus
cagtcgatcgatcgactgcgatactgtgctagcatgcatcgatcgaga

SEQ ID NO: 3 - DNA, 48 bp, Hepatitis C virus
cagtcgatcgatcgactgcgatagtgtgctagcatgcatcgatcgaga

SEQ ID NO: 4 - DNA, 48 bp, Hepatitis C virus
cagtcgatcgatcgactgcgattctgtgctagcatgcatcgatcgaga

SEQ ID NO: 5 - DNA, 12 bp, Artificial Sequence
  Synthetic
ttagtggaagcc